(19) United States
(12) Plant Patent Application Publication
(10) Pub. No.: US 2024/0122084 P1
(43) Pub. Date: Apr. 11, 2024

(54) PINEAPPLE PLANT NAMED 'FR4139'

(50) ;

(71) Applicant: Del Monte International GmbH, Zug (CH)

(72) Inventors: Joao Rodrigues, Limoeiro do Norte (BR); Hans Sauter, Coral Gables, FL (US); Ebrahim Firoozabady, Pleasant Hill, CA (US)

(73) Assignee: Del Monte International GmbH, Zug (CH)

(21) Appl. No.: 17/963,093

(22) Filed: Oct. 10, 2022

(51) Int. Cl. A01H 6/00

(52) U.S. Cl. PLT/258

(57) ABSTRACT

A new and distinct pineapple plant named ‘FR4139’, comprising certain unique traits, standing out Fusarium resistance obtained after crossing and backcrossing Fusarium resistant pineapple lines with an MD-2 Del Monte Gold pineapple. The invention incorporates stable, inheritable resistance to Fusariosis disease in a pineapple variety capable of addressing all of the high quality requirements of global export markets.

This application includes material that is subject to copyright protection. The copyright owner has no objection to the facsimile reproduction by anyone of the patent disclosure, as it appears in the Patent and Trademark Office files or records, but otherwise reserves all copyright rights whatsoever.

Genus and species: Ananas comosus.

Variety denomination: ‘FR4139’.

Inventors: Joao Rodrigues, Hans Sauter and Ebrahim Firoozabady.

### BACKGROUND OF THE NEW PLANT

The present invention comprises a new and distinct pineapple variety of Ananas comosus, hereinafter referred to as the variety named ‘FR4139’. The said variety has been developed by sexual crossing between plants of Manzana variety (not patented) and the variety ‘MD-2’ (non-patented), commercially known as ‘Del Monte Gold’, and selected from large populations of progenies of hybrids (F1), backcrossed three times (BC1, BC2 and BC3). This process started in 2009 in Limoeiro do Norte, Ceara, Brazil, and took thirteen consecutive years of iterative hybridization, selection, and planting. The new variety ‘FR4139’ in particular was the result of the back-cross of a selected plant of the ‘1/2.69’ line, with a selected plant of ‘MD-2’, to achieve the combination of characters of interest that qualify ‘FR4139’ as a variety of commercial potential.

The main objective of the hybridization program was to introduce stable resistance to Fusariosis disease of pineapples into a variety that would produce attractive fruits with excellent organoleptic traits and the right shape and size that would satisfy the strict requirements of export markets. The new cultivar ‘FR4139’ is being reproduced asexually faithful to the type in Limoeiro do Norte, Ceara, Brazil through the tissue culture technique. The new cultivar is being reproduced asexually faithful to the type in Limoeiro do Norte, Ceara, Brazil through the tissue culture technique.

### BACKGROUND INFORMATION ABOUT THE DISEASE

Among all the diseases of pineapple (Ananas comosus var. comosus), Fusariose caused by Fusarium guttiforme stands out for being the one that brings largest economic losses to prdoducers, with estimated losses between 50% and 100% of fruits and up to 50% in propagules (Matos, 2008).

Fusarium guttiforme can infect all parts of the pineapple causing the exudation of a gummy substance from the affected tissues. In plants, the lesion is located on the stem and progresses to the base of the leaf being restricted to the achlorophyllous region. Under field conditions an infected pineapple plant may show one or more external symptoms: stem tilt to the side where the lesion is located; changes in plant phyllotaxis, increasing the number of leaves per spiral; changes in the architecture of the plant that has the appearance of a funnel or cup; reduction in leaf length as well as in the overall development of the plant; “opening” of the central rosette of plants showing the youngest leaves; reducing the stem development; chlorosis; death of the apical meristem. As the disease progresses, the plant paralyzes growth and dies (Matos & Junghans, 2006).

In the fruits, the symptoms are manifested in the form of soft rot in the pulp, with accumulation of gum in the ovules of the ovary and exudation of the same through the infected floral cavity. At a more advanced stage of disease development, the skin of the fruit in the infected areas presents a red brown coloration and are at the lower level. These symptoms are observed with greater intensity in fruits near harvest (Matos & Junghans, 2006).

Following extensive field and laboratory research, four isolates of Fusarium guttiforme were identified for their particularly high virulence in the North East of Brazil, subsequently isolated and reproduced to serve as standard inoculum for the resistance trials the pineapple hybrids of the present invention were subjected to.

### SUMMARY OF THE INVENTION

The present invention is related to a new and distinct pineapple variety of the Bromeliaceae family, which was derived from a backcross (BC3) between the ‘MD-2’ and the ‘1/2.69’ (which in turn was already a backcross of a backcross between ‘MD-2’ X ‘Manzana’ cultivar), through hand pollination and mass selection process during thirteen years and provides Fusarium resistance to pineapple plants that could be grown in all areas, including where the disease caused by Fusarium guttiforme occurs.

In most characteristics, the selected hybrid is more alike to the ‘MD-2’ parent than the ‘1/2.69’ parent. The main difference is in the resistance to Fusarium guttiforme exhibited by the ‘FR4139’ and absent in the ‘MD2’ variety. The selected backcross 3 (BC3) plant's fruit has an internal quality and pulp color like the ‘MD-2’ fruit: the total soluble solid contents (TSS), citric acid, ascorbic acid, yellow pulp color, and the size of the fruit are like the ‘MD-2’. In addition, the ‘FR4139’ fruit is cylindrical in shape, and has a very pleasant aroma, and delectable taste, just like the ‘MD-2’ variety.

The new variety is characterized by occasional presence of spines at the leaf tip. Leaf color is gray-green and similar to the color of both parental lines, ‘MD-2’, and the BC2 ‘Manzana’ hybrid ‘1/2.69’ (FIG. 1).

### BRIEF DESCRIPTION OF THE PHOTOGRAPHS

The accompanying pictures illustrate the overall appearance of the new Ananas comosus var. comosus ‘FR4139’ showing infection damage caused by artificial inoculation with four different isolates of Fusarium guttiforme showing the colors as reasonably possible with colored reproductions of this type. The lesion caused by direct inoculation with an infected needle on the fruit helped verify the resistance trait (earlier established by immersion of young plants in a suspension of Fusarium guttiforme's most virulent isolate), when comparing reaction of fruit pulp tissue of the new pineapple variety ‘FR4139’ with ‘MD2’.

FIG. 1. View of the new variety ‘FR4139’ at 21.8 months after date of planting (17.2 months after planting (AP) plus 4.6 months after forcing (MAF).

FIGS. 2A-2B. Cross sectional view of ‘FR4139’ and ‘MD2’ fruit inoculated with Fusarium guttiforme isolates DM017, DMB35, DMB37 and DMB40 at 21.8 months DP and 4.6 MAF.

FIGS. 3A-3B. Longitudinal section view of Fusarium guttiforme inoculation site on fruit of ‘FR4139’ variety and ‘MD2’ fruit at 4.6 MAF.

FIGS. 4A-4B. External view of Fusarium guttiforme inoculation site on fruit of ‘FR4139’ and MD2 varieties at 4.6 MAF.

FIGS. 5A-5J. External view of inflorescence of ‘FR4139’ and MD2 varieties at 55 days after floral induction.

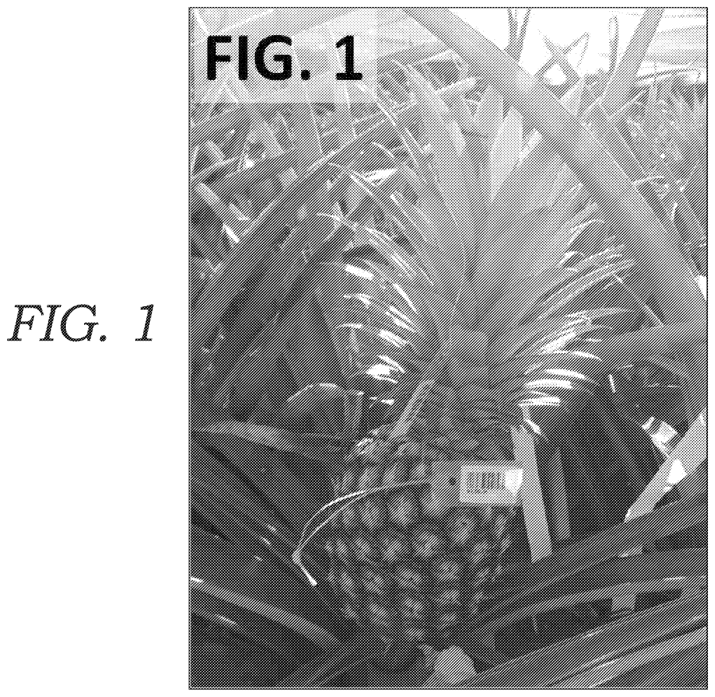

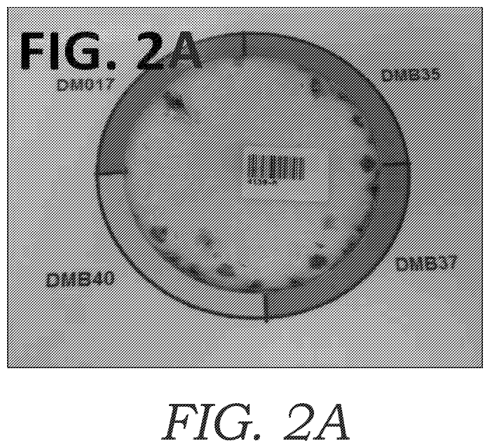

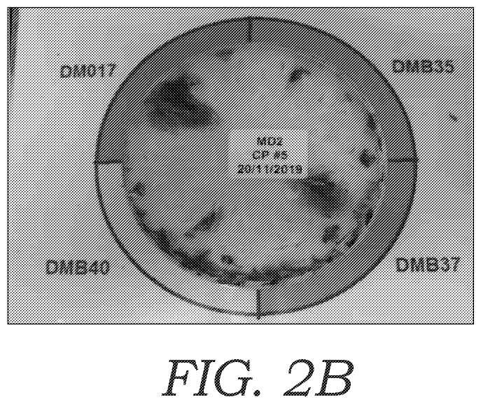

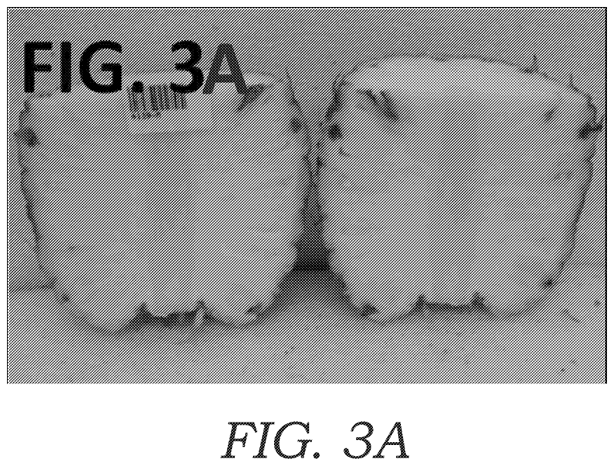

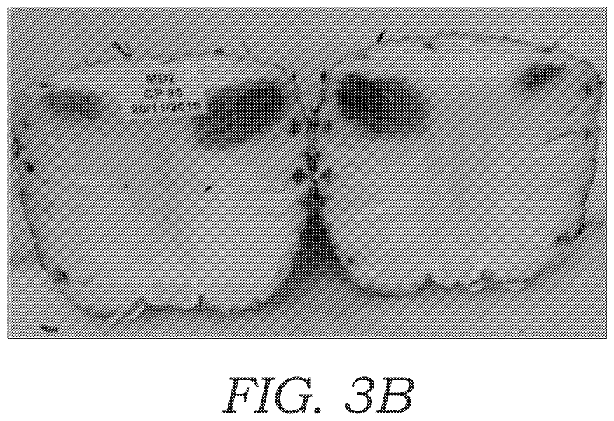

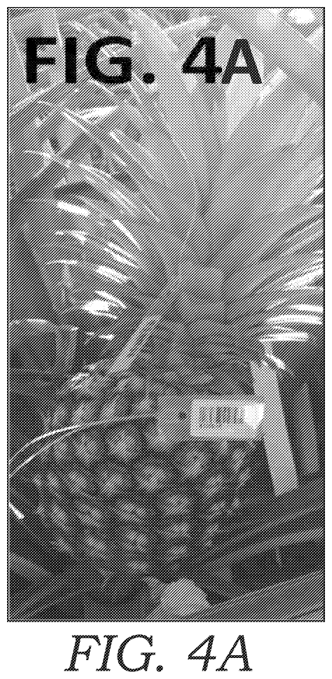

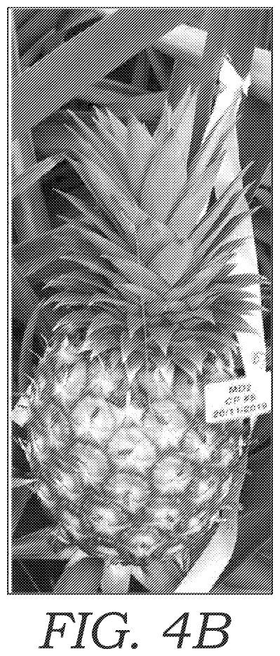

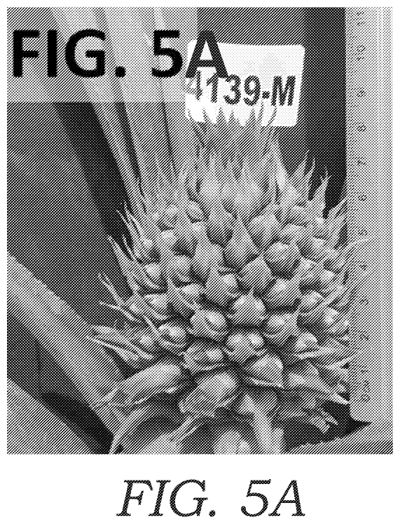

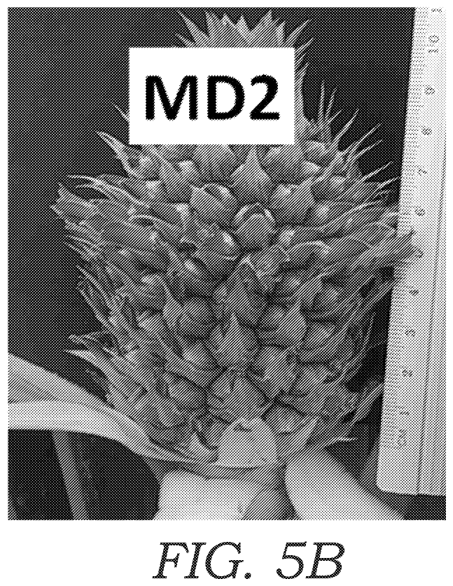

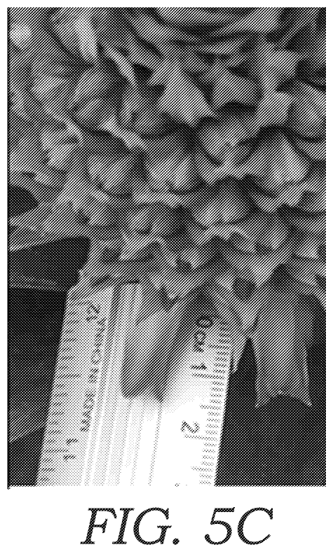

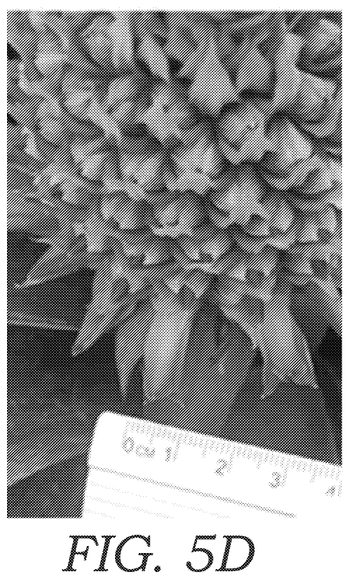

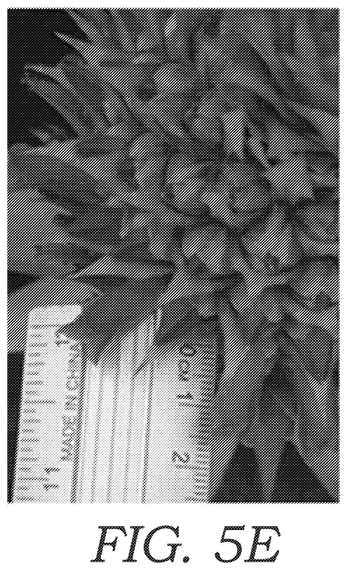

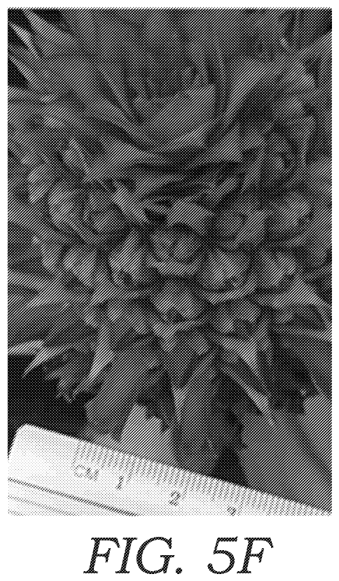

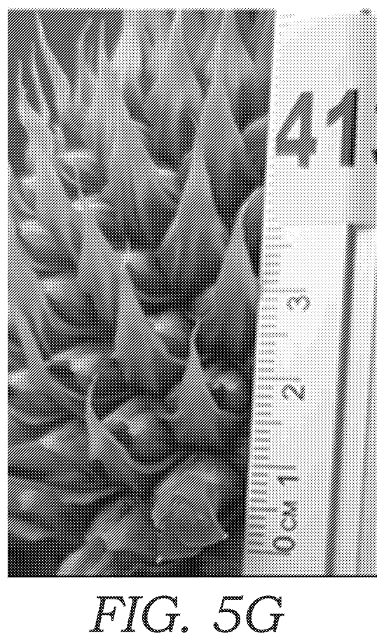

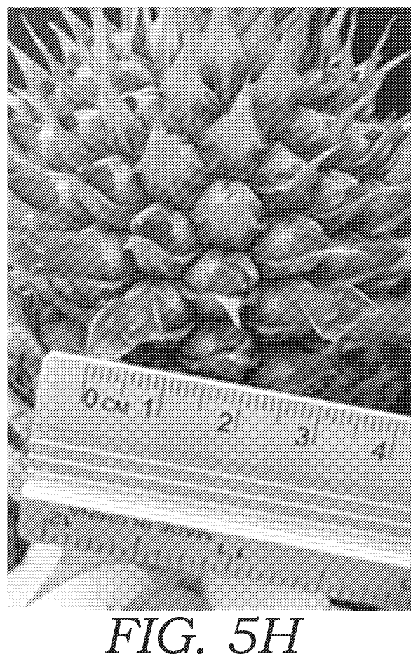

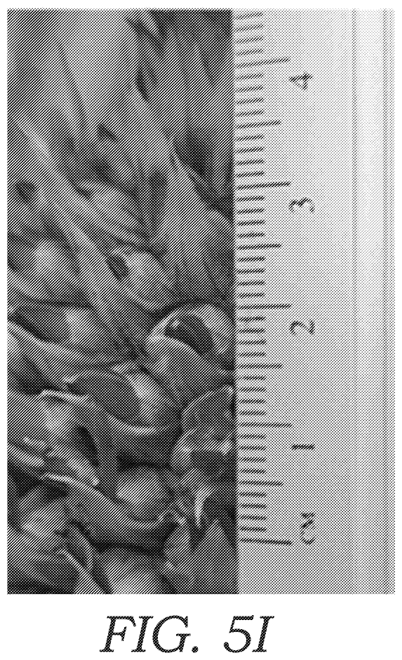

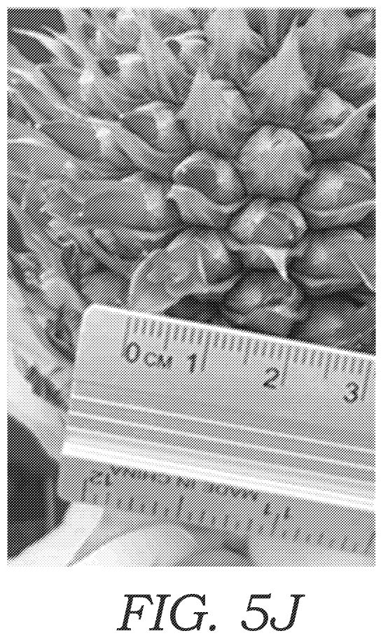

### DETAILED BOTANICAL DESCRIPTION

The new Ananas comosus cultivar ‘FR4139’ has not been observed under all possible environmental conditions. However, the plants were grown under environmental conditions and cultural practices which approximate those generally used in commercial pineapple growing operations. The phenotype of the new cultivar may still vary depending on the environmental conditions such as temperature, humidity, light intensity, and photoperiod without any change made to the genotype of the plant.

The aforementioned photographs, together with the following observations, measurements and values describing plants of ‘FR4139’ variety are based on observations made under optimally fertilized and growing conditions, in the region of Limoeiro do Norte, Ceará, Brazil (latitude−5.21945833° and longitude−37.914505°, where the temperatures generally range from 15.96-38.70° C., and an annual rainfall average of 746 mm.

The color terminology and designation reported here are in accordance to the Munsell Color Notation for Plants Tissues published by Munsell Color Macbeth, a division of Kollmorgen Corporation, Baltimore, Md. USA.

The following description was made based on a population of ‘FR4139’ plants, the BC3 hybrid obtained through hand pollination of parental lines in 2015 and planted in 2017 and fruit harvested in 2019 and new propagules produced from this plant planted in 2019.

Plant identification: Name: Ananas comosus var. comosus ‘FR4139’.

Parental Lines: Selected plant (BC2) ‘1/2.69’ backcrossed to ‘MD-2’.

Origin: Conventional genetic improvement (hand pollination), through crossing and backcrossing.

Classification: Botanical: Bromeliaceae or pineapple family. Subfamily: Bromelioidae. Genus: Ananas. Subgenus: comosus. Variety: ‘FR4139’.

Commercial: Bromeliad fruit plant (pineapple).

Form: Terrestrial (in plantation), with overlapping, sessile leaves from a funnel-formed rosette, surrounding a composite inflorescence (during anthesis), with few or no slips in the fruit peduncle and suckers that are produced in the stem and originate subsequent crops.
General Description: ‘FR4139’ (before anthesis).
   Stem.—I. General. Short, vertical, and covered by overlapping leaves, each leaf with a dominant axillary bud. II. Stem texture. Glabrous and fleshy, III. Stem size. A) Length (above soil level): usually between 17-28 cm at anthesis. B) Diameter between 6.30-9.20 cm at ground level to the anthesis. IV. Stem shape. Cylindrical and with a narrower diameter at the distal part.
   Leaves.—I. General. Closely overlapping sessile leaves (formed in acropetal succession) forming a dense rosette, the outline of which in longitudinal section is roughly heart shaped. The number of leaves fluctuates between 39-46 with a 5/13 phyllotaxy. II. Texture. A) Upper epidermal area: Glabrous, semirigid and channeled (or concave) except at the leaf tip. B) Lower epidermal area: Finely striated (longitudinally) and appears covered with a white layer consisting of scale like trichomes. III. Leaf arrangement. Alternate and in rosette shape. IV. Leaf margins. Flat, with rarely found irregularly spaced small deltoid cuspidate hooked spines usually located on the distal portions of leaves. V. Leaf venation. Parallel. VI. Leaf shape. Leaves are not uniform in shape and vary with the position of the leaf on the stem. The basal or oldest leaves are lanceolate while the base is considerably expanded. There is a noticeable narrowing in width between achlorophyllous (basal) and chlorophyllous (or main portion) of the leaves. The longest or most mature leaves are lanceolate in shape, but the base is without the arcuate expansions of the preceding leaves. The remaining leaves (or center leaves of the plant rosette) are lanceolate in form with no expansion of width into the base. VII. Leaf size (to anthesis). A) Length: Usually between 90.0-121.0 cm for those ‘D’ leaves with a non chlorophyllous base that usually is between 8.0-14.0 cm in length. B) Width: Normally between 3.0-7.8 cm in the mid leaf area of the longest leaves. The expanded basal disk usually has a maximum width of 4.8-11.0 cm. C) Thickness: In the longest leaves, it usually varies between 1.80-3.10 mm at the center of the mid leaf area and decrease laterally between 1.0-1.9 mm at the margin, while becoming slightly thinner towards the tip. The expanded basal disk at the mid stem area usually has a maximum thickness of 1.58-2.90 mm at the center of the blade and tapering laterally toward margins up to 0.30-0.95 mm. VIII. Leaf color like ‘MD2’. A) Upper epidermal surface: 1. General: dominant color is usually dark green. The color of the basal disk is predominantly white and light green. achlorophyllous basal disk area: commonly pale white. Mid leaf area: commonly dark green (7.5GY 4/6). Leaf tip area: commonly dark green (7.5GY 4/6). B) Lower epidermal surface (underside): General: commonly green to grayish green (10GY 5/2) with pale white basal disk area (N8).
   Inflorescence (at anthesis).—I. General. Flower composite from 49-89 fruitlets borne per inflorescence of a long peduncle of approximately 5.0-5.5 cm length at the apical meristem. Individual bisexual flowers that consist of three Sepals, Six Stamens, three Stigmas and three Carpels. The inflorescence is self-incompatible producing edible fruit parthenocarpically (production of fruit without fertilization of ovules). II. Texture. Glabrous and fleshy. III. Shape. Oval with slightly raised flowers with a light red to grayish red color in the crown. IV. Size and color. Comparable to specimens of Ananas comosus var. comosus. Petal size: 1.60 cm. Petal color in the apex: light purple (10P 5/18). V. Sepal size. 0.54 cm. Sepal color: reddish brown (5R 5/6). VI. Floral bract's length. From 1.97 cm, serrated margins (with tiny spines); reddish color (5R 5/14).
   Crown (at harvest).—I. General. Visually one crown. Crown leaves are short and erect at anthesis. II. Leaf arrangement. Alternate and in rosette shape. III. Leaf margins. Flat with smooth borders. Seldom very small spines in the tip of one leaf IV. Size at harvest. Average 121.7 cm. Weight: average 317.2 g V. Shape. Medium-big crown with medium width and semirigid leaves. VI. Color of the terminal crown leaves: Dark green color at the apex (5GY 3/6) and dark green at the base (5GY 3/6).
   Fruit (at harvest).—I. Size. Usually with a weight between 1249-1649 grams and average weight of 1419 grams. Fruit core's diameter 2.06 cm. II. Shape. Cylindrical with flat and medium size fruitlets. Medium-big crown with thin and semirigid leaves. Average fruit's height 12.7 cm, fruit's diameter: average 11.0 cm. III. How borne. The development of the fruit occurs from the apical meristem of the plant on a long peduncle, usually between 5.0-13.7 cm in length. IV. Color. A) Shell: commonly grayish green at early maturity (10GY 5/2), with yellowish brown bract (5Y4/2). Fruit with yellow peduncle (2.5Y 7/6). B) Pulp: Usually yellow (2.5Y 7/6). V. Brix. Typically, average 15.6 degrees, standing out from their parents. VI. Total acid levels. Usually between 0.64-0.96 milligrams of citric acid/ml of juice, with an average of 0.84 mg/ml. VII. Vitamin C content reported as ascorbic acid content. Regularly between 38.66 and 60.04 mg/100 ml of juice, with an average of 47.80 mg/100 ml. VIII. Plant/fruit resistance/susceptibility to pest and diseases. The plant of the new variety ‘FR4139’ performs very similar to ‘MD-2’ differentiating mainly in its resistance to Fusarium guttiforme. Table 2 shows the comparison of pineapple varieties ‘FR4139’, ‘MD-2’ and ‘Champaka’ (not patented) and their resistance to Fusarium guttiforme.
   Others.—I. Fertility. This plant is self-incompatible. This is the reason why the presence of sexual seeds is almost null. The material used for planting are suckers, fruit crowns and vitroplants. II. Vigor. This plant exhibits similar vigor as its parents, the ‘1/2.69’ line, and the ‘MD-2’ variety. III. Yield. A population of this pineapple can have an agronomic yield from 83 to 164 ton/ha. IV. Plant use. The fruit will be commercialized within the fresh fruit and processed fruit for the export markets.
   Summary of the Special Characteristics.—The ‘FR4139’ plants present the following differences when compared to its parental lines: A. Resistance to Fusarium guttiforme; MD-2 is very susceptible to this pathogen, while ‘FR4139’ exhibits total resistance. B. Yellow colored pulp: the backcross between the ‘1/2.69’ backcross and ‘MD-2’, ended up in a combination of a fruit with similar shell color and yellow pulp like the ‘MD-2’ variety. C. Plant with little or none slips: ‘MD-2’ variety produces between 1-3 slips per plant, and the ‘1/2.69’ produces between 0-5 slips per plant. By comparison, ‘FR4139’, which reduces cosmetic and pest problems that can be originated by the contact of the slips with the fruit. D. At full maturity, the ‘FR4139’ fruit achieves high sugar content, as measured by brix levels. Citric acid, and ascorbic acid are similar as those of ‘MD-2’ and ‘1/2.69’ fruits. E. As a result of the backcross process, ‘FR4139’ has acquired resistance to Fusarium guttiforme, just like the ‘1/2.69’ but with an improved fruit size, pulp color, and internal quality similar to the ‘MD-2’. This combination of characteristics is important to differentiate this hybrid from its parents; as shown on Table 3, when reproduced asexually, these characteristics are stable and reproducible for successive generations.

TABLE 1
Average data for select organoleptic parameters and fruit size of the ‘FR4139’ hybrid.
(data for ‘FR4139’ represent observations from the first plant evaluated when backcrossing ‘MD-
2’ with 1/2.69 hybrid; data for ‘MD2’ represents the average score for ‘MD2’ variety in the region).

| | | | Ascorbic. | Citric | Fruit | |
|---|---|---|---|---|---|---|
| | | | Acid | Acid | Weight | Number |
| Generation | n | Brix | (m g/100 ml) | (mg/ml) | (g) | of slips |
| 'FR4139’ | 1 | 16.6 | 41.8 | 0.96 | 1509 | 0 |
| 'MD2’ | 71 | 16.3 | 61.4 | 0.95 | 1949 | 0 |

Individual plant description: The following is an overview of ‘FR4139’ hybrid, a new pineapple plant variety, that was developed through hybridization process. in Limoeiro do Norte, Ceará, Brazil.
   Plant Age.—17.2 months after planting plus 4.6 months after forcing.
   Plant growth habit.—Semi-upright.
   Plant diameter.—Around 159.00 cm between opposite leaf tips.
   Plant height.—115.0 cm above ground surface.
   Stem.—Length. Between 17.20-28.40 cm above ground sur-face. Diameter. Between 7,33-9.28 cm above ground surface.
   Leaves.—I. Number. 58 leaves. II. Length. 112 cm the longest leaf. III. Width. (Largest leaves) at mid leaf(max.) 7.0 cm. Leaf piping is absent. IV. Thickness. 2.79 mm along the axis.

V. Color: A) Upper epidermal chlorophyllous area: Commonly dark green 7 (7.5GY 4/6). B) Upper epidermal non chlorophyllous area: commonly pale white (N8). C) Lower epidermal area: Commonly grayish green (10GY 5/2). Leaf anthocyanin coloration may appear slightly on some plants.
Inflorescence.—General: Composite inflorescence borne on a long peduncle at the apical meristem. The inflorescence is composed of 76 flowers, which eventually lead to an equal number of fruitlets.

I. Petals: Description of size and texture of flowers of ‘FR4139’ hybrid.

| Inflorescence | UPOV ID | Trait | Note |
|---|---|---|---|
| | 14 | Size of bracts (cm) | 1.97 |
| | 15 | Petal: color of apex | 2 |
| | | (purple red), | |
| | | base (white) | |
| | 16 | Petal length (mm) | 16.0 |
| | N/A | Petal width (cm) | 0.80 |
| | N/A | Petal Height | 1.60 |
| | N/A | Petal texture | very finely textured |
| | | | and smooth feel |

II. Reproductive organs. Description of size and shape of reproductive organs of ‘FR4139’ hybrid.

| | Trait | Note |
|---|---|---|
| Inflorescence | Style (mm) | 12.52 |
| | Stamen (mm) | 10.48 |
| | Style shape | tubular |
| | Stamen shape | tubular |
| | Style color | Base = white, top-pale purple |
| | Filament color | Filament base = white, filament |
| | | top-pale purple |
| | Anther color | Pale yellow |
| | Pollen color | transparent |

III. Peduncle. length and diameter of the peduncle of ‘FR4139’ hybrid are as follows.

| Peduncule | UPOV ID | Trait | Note |
|---|---|---|---|
| | 21 | Length (cm) | 11.8 ± 6.8 |
| | 22 | Diameter (cm) | 2.5 ± 0.7 |

TABLE 2
Susceptibility to pest and diseases of different commercial
varieties to Fusarium guttiforme (‘FR4139’ is
resistant and ‘Champaka’ and ‘MD2’ are susceptible).

| | Pineapple Variety | | |
|---|---|---|---|
| Pest/Disease | ‘FR4139’ | ‘MD-2’ | ‘Champaka’ |
| Fusarium guttiforme. | R+ | S + | S |

The pineapple variety ‘FR4139’ has a post-harvest shelflife similar to ‘MD-2’ variety, showing similar performance in shell dehydration studies.

‘FR4139’ fruit general characteristics are as follows (A) a fruitlet apex which is flat; (B) the flesh density is medium; (C) the number fruitlets per syncarp is 47-93; (D) the fruitlet characteristics are as follows:

| | UPOV ID | Trait | Note |
|---|---|---|---|
| Fruit | 31 | Fruit shape (cylindrical) | 3 |
| | 32 | Fruit length (cm) | 12.9 ± 1.6 |
| | 33 | Fruit diameter (cm) | 10.9 ± 1.0 |
| | 34 | Predominant color of mature | 6 |
| | | eye (medium yellow) | |
| | 35 | Fruit: Size (medium) | 5 |
| | N/A | Size of eye diameter width (cm) | 2.38 ± 0.9 |
| | N/A | Size of eye length (cm) | 2.38 ± 0.9 |
| | N/A | Color of immature eye (grayish green) | 4 |
| | 37 | Fruitlet apex (flat) | 2 |
| | 38 | Evenness of color of eyes | 1 |
| | | (even or slightly uneven) | |

IV. Shell color. ‘FR4139’ shell color at mature (10YR 6/8) and immature (2.5GY 2/4) stages.
V. Weight and shape of fruit. ‘FR4139’ average fruit weight is similar to the ‘MD-2’, and ‘Champaka’; ‘FR4139’ has a cylindrical shape.
VI. Ascorbic acid.'FR4139′ has a higher content of ascorbic acid than its relative A. comosus cv. ‘Manzana’, but similar to ‘MD-2’ variety.
VII. Citric acid.'FR4139′ citric acid content is similar to ‘MD-2’.
VIII. Brix.The sugar content (measured as brix degrees) of ‘FR4139’ is slightly higher to that found on ‘MD-2’.
IX. Age to forcing. ‘FR4139’ is vigorous and can reach forcing plant size 8-17 months after planting. Although this depends on the planting material used and the desired plant weight for floral induction, under similar conditions, ‘MD-2’ reaches an optimal forcing size 8.5 months after planting.
X. Leaf spines. This characteristic is commonly used to differentiate pineapple plants from other varieties. The color of the leaf spines are as follows: Tip: dark green like MD2 (7.5GY 4/6), base: dark green like MD2 (7.5GY 4/6). ‘FR4139’ does not have conspicuous or regular thorns on the leaf like its parental ‘1/2.69’. It does parental ‘MD-2’, although ‘MD-2’ often presents irregular thorns on the edges of the leaf blade as well.
XI. Fruit quality of ‘FR4139’ when compared with other pineapple varieties: most pineapple varieties grown worldwide are produced for local consumption, and very few are grown for international commercialization and global distribution. The fruit characteristics bread into ‘FR4139’ were specifically chosen to comply with the strictest quality standards of export markets like those of North America, Europe, and Asia Pacific. Hence, all of the relevant data about ‘FR4139’ reported here is compared against ‘MD-2’, the pineapple variety that has achieved most notoriety to date in global markets.

TABLE 3
Comparison of ‘FR4139’ against other varieties on some of the most important
characteristics relevant to fresh fruit destined for exportation.
Comparative characteristics of different pineapple varieties and cultivars

| Variety/ | # Of slips | | Fruit weight (g) | | Ascorbic Acid (mg/100 ml) | |
|---|---|---|---|---|---|---|
| Cultivar ⁽¹¹⁾ | Average | Range | Average | Range | Average | Range |
| ‘FR4139’⁽¹⁾ | 0 | 0-1 | 1361 | 1124-1649 | 47.80 | 38.66-60.40 |
| ‘MD2’⁽²⁾ | 1.2 | 0-3 | 1820 | 1070-2560 | 53.06 | 37.00-69.06 |
| ‘Morada’⁽³⁾ | 7.57 | 4-9 | 1887 | 1566-2000 | 20.03 | 9.90-24.90 |
| ‘Honey Gold’ | 1.5 | 0-3 | 1033 | 450-1678 | 21.14 | 14.73-37.36 |
| ‘Champaka 153’ ⁽³⁾ | 1.1 | | 1710 | 420-3010 | 12.91 | 8.10-17.72 |
| ‘Champaka F152’ | 1.5 | | 2328 | | | |
| ‘CO-2’⁽⁴⁾ | | 2-3 | 2059 | 1297-2590 | | 30.80-55.50 |
| ‘Singapore’ | | 2-12 | 1000 | | | |
| ‘Spanish ⁽⁵⁾ | | | | | | |
| ‘Sarawak’⁽⁵⁾ | 0 | | | 2000-4000 | | |
| ‘Mauritius’ ⁽⁵⁾ | 0 | | | 500-1500 | | |
| ‘Josephine’⁽⁶⁾ | | | | 1100-1300 | | |
| ‘Scarlett’⁽⁶⁾ | | | | 1400-2000 | | |
| ‘Red Spansh’ ⁽⁷⁾ | | 1-3 | | 1200-2000 | | |
| ‘T'ainung 11’⁽⁷⁾ | 6.9 | | 991 | 733-1269 | | 1.40-18.50 |
| ‘Imperial’⁽⁸⁾ | 9 | | 1792 | | | |
| ‘Perolera’⁽⁸⁾ | | 8-10 | 1800 | | | |
| ‘Pernambuco’⁽⁹⁾ | | | | 1000-1500 | | |
| ‘Primavera’⁽⁹⁾ | | 7-10 | 1300 | | | |
| ‘Queen’⁽¹⁰⁾ | 10 | | | 500-1000 | 26 | |

| Variety/ | Citric Acid (mg/ml) | | °Brix | |
|---|---|---|---|---|
| Cultivar ⁽¹¹⁾ | Average | Range | Average | Range |
| ‘FR4139’⁽¹⁾ | 0.74 | 0.54-0.96 | 16.10 | 13.8-18.1 |
| ‘MD2’⁽²⁾ | 0.6 | 0.36-0.84 | 15.55 | 12.9-17.2 |
| ‘Morada’⁽³⁾ | 0.69 | 0.58-0.86 | 13.51 | 12.2-15.1 |
| ‘Honey Gold’ | 0.98 | 0.67-1.33 | 16.18 | 14.4-18.1 |
| ‘Champaka 153’ ⁽³⁾ | 0.72 | 0.54-0.90 | 14.33 | 11.6-17.0 |
| ‘Champaka F152’ | 0.73 | | 14.97 | |
| ‘CO-2’⁽⁴⁾ | | 0.42-0.91 | | 15.0-16.7 |
| ‘Singapore’ | | 0.50-0.60 | | 10.0-12.0 |
| ‘Spanish’⁽⁵⁾ | | | | |
| ‘Sarawak’⁽⁵⁾ | | 0.30-0.65 | | 14.0-17.0 |
| ‘Mauritius’⁽⁵⁾ | | 0.40-0.60 | | 15.0-17.0 |
| ‘Josephine’⁽⁶⁾ | | | | 17.0-22.0 |
| ‘Scarlett’⁽⁶⁾ | | | | 15.0-18.0 |
| ‘Red Spansh’⁽⁶⁾ | | | 12.00 | |
| ‘T'ainung 11’⁽⁷⁾ | 0.5 | 0.40-0.6 | 14.00 | 13.2-15.1 |
| ‘Imperial’⁽⁸⁾ | 0.62 | | 15.80 | |
| ‘Perolera’⁽⁸⁾ | 0.64 | | 13.10 | 14.0-16.0 |
| ‘Pernambuco’⁽⁹⁾ | | | | |
| ‘Primavera’⁽⁹⁾ | 0.51 | | 13 | |
| ‘Queen’⁽¹⁰⁾ | 0.56 | | | 14.0-16.0 |

⁽¹⁾FR4139 fruit harvested in Brazil.
⁽²⁾Pindeco's historical data base and monthly research report April 2001.
⁽³⁾Pindeco's fruit historical data base. Pindeco's forcing plant weight data base.
⁽⁴⁾Plant patent 8,863.
⁽⁵⁾Wee, Y. C. 1972. Some common pineapple cultivars of west Malaysia. Malays Pineapple pp 7-13.
⁽⁶⁾Bartholomew et al. 2003 The Pineapple, Botany, Production and Uses.
⁽⁷⁾Chang, Ching-Chyn, 1995 Tainung No. 13. Pineapple. Jour. Agric. Res. China 44(2): 287-296.
⁽⁸⁾Pinto da Cunha et al. 0 abacaxizeiro. Pineapple News Issue No 10 May 2003.
⁽⁹⁾Pinto da Cunha et al. 0 abacaxizeiro. Py et al. The pineapple Cultivation and uses.
⁽¹⁰⁾Del Monte pineapple germplasm collection database.
⁽¹¹⁾Cultivars are unpatented unless indicated otherwise.

### REFERENCES CITED

MATOS, A.P. (2008) - Perdas causadas pela F. guttiforme. Disponivel em: https://www.embrapa.br/busca-de-publicacoes/-/publicacao/981520/situacao-e-perspectivas-da-abacaxicultura-no-amazonas

MATOS, A. P. & JUNEGHANS, D. T. Variedades de abacaxi resistentes a F. guttiforme. 2006. Cruz das Almas, BA. https://ainfo.cnptia.embrapa.br/digital/bitstream/item/42932/1/VARIEDADES-ABACAXI-ARISTOTELES.pdf.

Munsell Value and Color Conver. Android App. Created in 2021, version 3 in 2021.

What is claimed is:

1. A new and distinct Ananas comosus plant named ‘FR4139’, as illustrated and described herein.
